(19) United States
(12) Patent Application Publication
(10) Pub. No.: US 2011/0190641 A1
(43) Pub. Date: Aug. 4, 2011

(54) BIOLOGICAL INFORMATION MEASUREMENT APPARATUS

(76) Inventors: Kiyoshi Tateishi, Kanagawa (JP); Yoshinori Kimura, Kanagawa (JP)

(21) Appl. No.: 13/061,079

(22) PCT Filed: Aug. 28, 2008
(86) PCT No.: PCT/JP2008/065413

§ 371 Date: Apr. 8, 2011

(51) Int. Cl. A61B 6/00

(52) U.S. Cl. 600/479

(57) ABSTRACT

Disclosed herein is a biological information measurement apparatus for rendering laser light incident on an examinee and measuring a state of internal tissue of the examinee based on light scattered within the examinee. The biological information measurement apparatus includes a laser light source for emitting the laser light, photoelectric conversion means for receiving the scattered light and generating a measurement signal based on the scattered light, signal amplification means for generating an amplified signal by amplifying a signal level of the measurement signal, signal supply means for intermittently supplying the measurement signal to the signal amplification means, first output means for intermittently holding the amplified signal corresponding to a period in which the measurement signal is supplied to the signal amplification means and outputting the held signal as a first signal, second output means for intermittently holding the amplified signal corresponding to a period in which the measurement signal is not supplied to the signal amplification means and outputting the held signal as a second signal, signal subtraction means for generating a subtraction signal based on a difference between the first signal and the second signal, and arithmetic output means for arithmetically outputting information about the internal tissue of the examinee based on the subtraction signal.

### TECHNICAL FIELD

The present invention relates to a biological information measurement apparatus which renders laser light incident on the surface of biological tissue and detects a blood flow, etc. in the biological tissue based on light scattered therein.

### DESCRIPTION OF THE RELATED ART

The blood flow measurement principle of a blood flow sensor using laser light is as follows. Laser light is projected on tissue through an optical fiber for laser irradiation connected to a laser diode. The laser light is almost semi-spherically propagated while being repeatedly scattered and reflected by blood cells in capillaries or the tissue. Light scattered in the tissue is received by an optical fiber for light reception and then converted into an electrical signal by a photodiode connected to the light reception fiber. At this time, light scattered from a moving blood cell generates a frequency shift by the Doppler effect in proportion to a traveling speed of the blood cell. The difference between the frequency of the light scattered from the static tissue and the frequency of the light scattered from the moving blood cell is distributed over about a band of about several hundred Hz to several tens of KHz, and a bit signal generated by interference between the two lights is thus sufficiently detectable. In a power spectrum of this bit signal, a Doppler shift frequency corresponds to the speed of the blood cell and power corresponds to the amount of the blood cell. A blood flow is a total sum of products of the speeds of respective blood cells and the number of the blood cells. As a result, the blood flow can be obtained by obtaining power spectra of bit signals, multiplying the obtained power spectra by frequencies and adding up the multiplication results.

FIG. 1 is a block diagram schematically showing the configuration of a conventional blood flow sensor. A laser driving circuit 100 supplies light emission drive current to a laser diode 101. The laser diode 101 emits laser light of power based on the drive current. The laser light is projected on a human body or the like, which is an examinee. The laser light is scattered within the examinee and the reflected, scattered light is received by a photodiode 102. The photodiode 102 performs photoelectric conversion for the scattered light to generate an optical detection signal based on the intensity of the light. Because the signal component of the optical detection signal is weak, the signal level thereof is amplified by an amplifier 103. An analog/digital (AD) converter 104 converts the amplified measurement signal into a digital signal. A signal processing circuit 105 performs signal processing for the digital signal, performs a frequency analysis of an interference component of the scattered light to calculate a blood flow, and outputs the calculation result of the blood flow to an output unit 106 through an interface.
Patent Literature 1: Japanese Patent Kokai No. 2007-167369

### SUMMARY OF THE INVENTION

### Problems to be Solved by the Invention

As mentioned above, light scattered in the examinee is converted into an electrical signal and output as an optical detection signal by the photodiode. Because this optical detection signal is weak, it is amplified by the amplifier. The signal component of the optical detection signal output from the photo detector is a low-frequency signal component. For this reason, noise in a low frequency domain of the amplifier, namely, 1/f noise needs to be addressed. The 1/f noise has a characteristic that it increases in inverse proportion to frequency. The 1/f noise is considered to be generated as a trap of a gate oxide film of a metal oxide semiconductor (MOS) transistor constituting the amplifier, which originates from an impurity or crystal defect of the gate oxide film, replenishes/discharges carriers at random. As this noise component increases in the output signal of the amplifier, measurement precision decreases. Also, in the case of a large noise component, when the gain of the amplifier is set to a high value, it may exceed an output dynamic range of the amplifier, resulting in the signal component being saturated. In order to cope with this problem, a supply voltage to the amplifier may be raised to enlarge the output dynamic range. In this case, however, the gain of the amplifier may exceed an input dynamic range of the downstream AD converter, resulting in digital data after quantization being saturated. Conversely, when the gain of the amplifier is set to a low value so as not to exceed the input dynamic range of the AD converter, the signal component is degraded, thereby making it impossible to secure detection precision. In this case, there is no choice but to use a costly high-resolution AD converter. As stated above, provided that a signal with a large noise component is output from the amplifier, measurement precision will be deteriorated and there will be difficulty in processing the signal. Accordingly, it is preferable to remove only a noise component overlapping a measurement signal.

The present invention has been made in view of the above problems, and it is an object of the present invention to provide a biological information detection apparatus which is capable of removing only a noise component contained in a measurement signal, so as to realize high detection precision.

### Means for Solving the Problems

A biological information measurement apparatus according to the present invention is a biological information measurement apparatus for projecting laser light on an examinee and measuring a state of internal tissue of the examinee based on light scattered within the examinee, the apparatus including a laser light source for emitting the laser light, photoelectric conversion means for receiving the scattered light and generating a measurement signal based on the scattered light, signal amplification means for generating an amplified signal by amplifying a signal level of the measurement signal, signal supply means for intermittently supplying the measurement signal to the signal amplification means, first output means for sampling the amplified signal corresponding to a period in which the measurement signal is supplied to the signal amplification means and outputting the sampled signal as a first signal, second output means for sampling the amplified signal corresponding to a period in which the measurement signal is not supplied to the signal amplification means and outputting the sampled signal as a second signal, signal subtraction means for generating a subtraction signal based on a difference between the first signal and the second signal, and arithmetic output means for arithmetically outputting information about the internal tissue of the examinee based on the subtraction signal.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram showing the configuration of a conventional blood flow sensor;

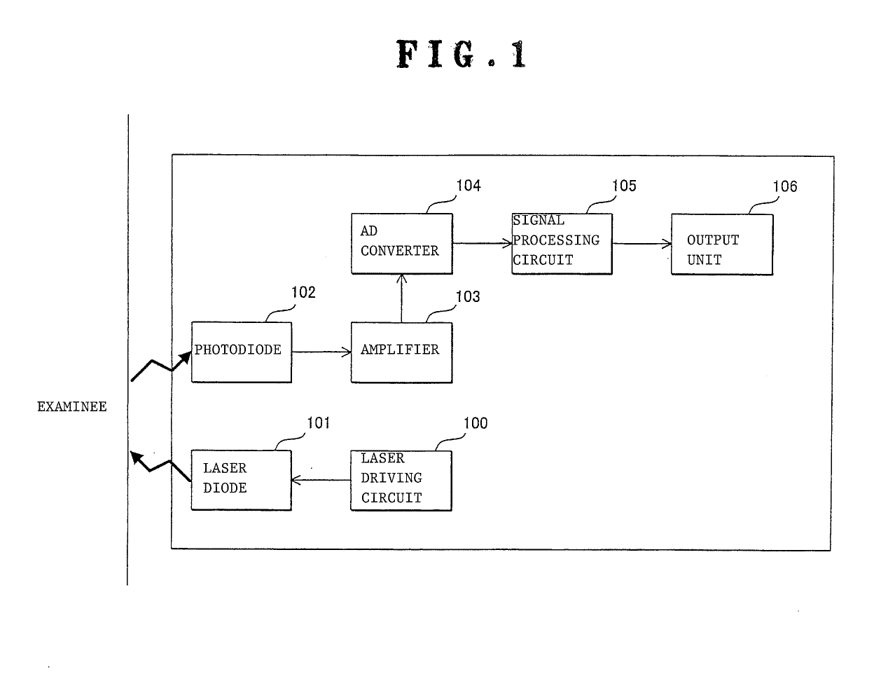

FIG. 2 is a block diagram showing the configuration of a blood flow sensor according to an embodiment of the present invention;

FIG. 3 is a block diagram showing the configurations of a photodetector, a switch and an I-V converter according to an embodiment of the present invention;

FIG. 4 is a block diagram showing the configurations of sample/hold circuits according to an embodiment of the present invention;

FIG. 5 is a block diagram showing the configuration of a subtracter according to an embodiment of the present invention;

FIG. 6 is a timing chart illustrating the operation of a blood flow sensor according to an embodiment of the present invention;

FIG. 7 is a block diagram showing the configuration of a blood flow sensor according to another embodiment of the present invention;

FIG. 8 is a timing chart illustrating the operation of a blood flow sensor according to the embodiment of the present invention;

FIG. 9 is a block diagram showing the configuration of a blood flow sensor according to another embodiment of the present invention;

FIG. 10 is a timing chart illustrating the operation of a blood flow sensor according to another embodiment of the present invention;

FIG. 11 is a block diagram showing the configuration of a blood flow sensor according to another embodiment of the present invention;

FIG. 12 is a block diagram showing the configuration of a blood flow sensor according to another embodiment of the present invention;

FIG. 13 is a block diagram showing the configuration of a switch according to another embodiment of the present invention;

FIG. 14 is a block diagram showing the configuration of a switch according to another embodiment of the present invention;

FIG. 15 is a block diagram showing the configuration of a switch according to another embodiment of the present invention;

FIG. 16 is a block diagram showing the configuration of a blood flow sensor according to another embodiment of the present invention;

FIG. 17 is a block diagram showing the configuration of a pulse driving circuit according to another embodiment of the present invention;

FIG. 18 is a view illustrating an I-P characteristic of a semiconductor laser;

FIG. 19 is a timing chart illustrating the operation of a blood flow sensor according to another embodiment of the present invention; and

FIG. 20 is a block diagram showing the configuration of a pulse driving circuit according to another embodiment of the present invention.

### BEST MODE FOR CARRYING OUT THE INVENTION

Reference will now be made in detail to the preferred embodiments of the present invention, examples of which are illustrated in the accompanying drawings. Wherever possible, the same reference numbers will be used throughout the drawings to refer to the same or like parts.

### First Embodiment

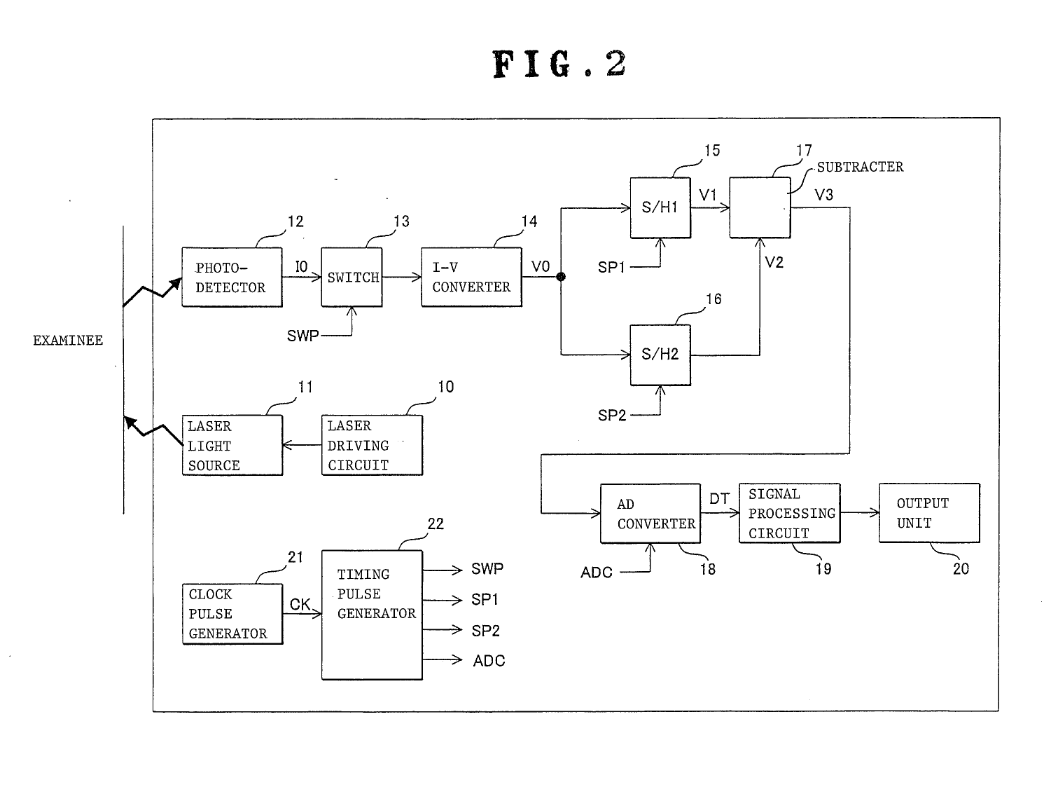

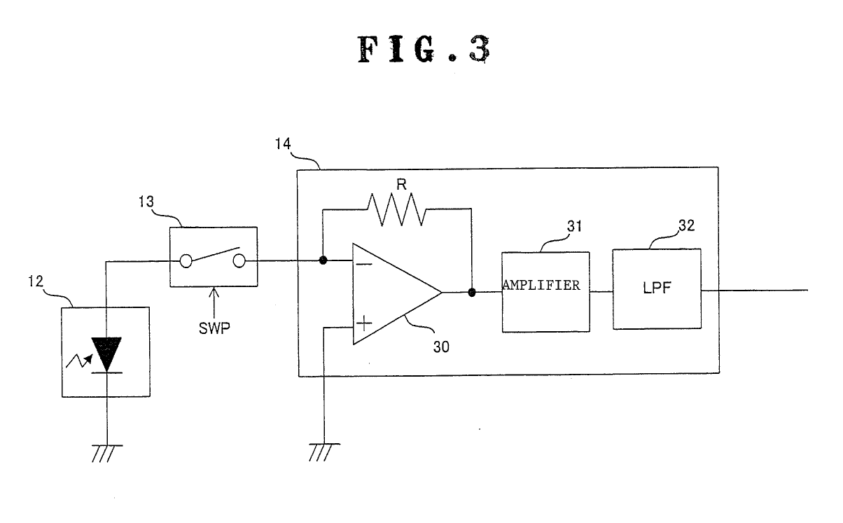

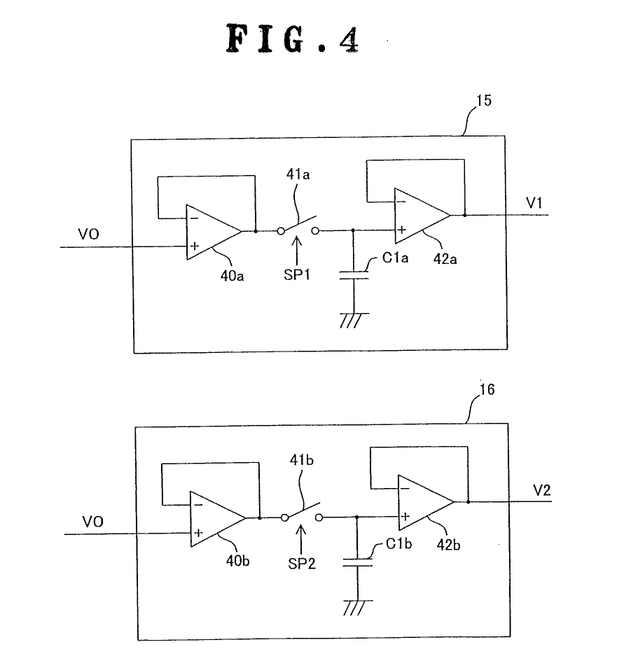

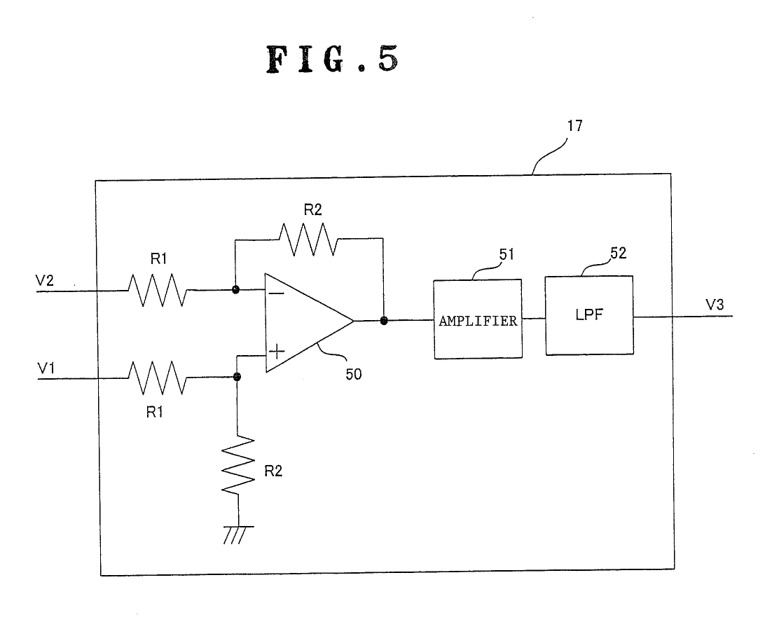

FIG. 2 is a block diagram showing the configuration of a blood flow sensor according to an embodiment of the present invention, FIG. 3 is a block diagram showing in detail the configurations of a photodetector 12, a switch 13 and a current to voltage (I-V) converter 14 constituting the blood flow sensor, FIG. 4 is a block diagram showing in detail the configurations of sample/hold circuits 15 and 16 of the blood flow sensor, and FIG. 5 is a block diagram showing in detail the configuration of a subtracter 17 of the blood flow sensor.

A laser driving circuit 10 generates drive current to light a laser light source 11, and supplies it to the laser light source 11. For example, a semiconductor laser may be used as the laser light source 11. The laser light source 11 emits laser light of output power based on the drive current supplied from the laser driving part 10.

The photodetector 12 may include, for example, a PIN photodiode, etc. The photodetector 12 generates optical detection current T0 based on the intensity of light incident on a PN junction. Also, optical waveguides may be formed between the laser light source 11 and photodetector 12 and an examinee by connecting optical fibers to the laser light source 11 and photodetector 12.

The switch 13 may include, for example, a complementary metal oxide semiconductor (CMOS) circuit, and is disposed between the I-V converter 14 and the photodetector 12. In the switch 13, a transistor is turned on/off based on a switch control signal SWP supplied from a timing pulse generator 22 to perform a switching operation. The optical detection current I0 is supplied to the I-V converter 14 when the switch circuit 13 is on, and is not supplied to the I-V converter 14 when the switch 13 is off.

The I-V converter 14 may include, for example, an operational amplifier 30 having input and output terminals between which a feedback resistor R (resistance R) is connected, an amplifier 31, and a low pass filter 32, as shown in FIG. 3. The operational amplification circuit 30 has an inverting input terminal connected to one terminal of the switch 13, and a non-inverting input terminal fixed at ground potential. The operational amplification circuit 30 converts the optical detection current I0 supplied through the switch 13 into a voltage signal having a voltage level of −R·IO by allowing the optical detection current I0 to flow to the feedback resistor R. This voltage signal is amplified by −K times by the amplifier 31 and is then passed through the low pass filter 32, so that an unnecessary high-frequency component is removed therefrom. In other words, the I-V converter 14 converts the input optical detection current I0 into a voltage signal having a voltage level of K1·R·I0 and outputs the converted voltage signal as an I-V-converted signal V0. As a result, a weak signal level of the optical detection current I0 is amplified. In the case where general MOS transistors constitute the operational amplifier 30, etc., 1/f noise generated by the operational amplifier 30 itself overlaps the output I-V-converted signal V0. The I-V-converted signal V0 output from the I-V converter 14 is supplied to the first and second sample/hold circuits 15 and 16.

Each of the first and second sample/hold circuits 15 and 16 includes, as shown in FIG. 4, a voltage follower 40a or 40b and a voltage follower 42a or 42b provided respectively at an input side and an output side of the corresponding sample/hold circuit, an analog switch 41a or 41b having one terminal connected to an output terminal of the voltage follower 40a or 40b at the input side, and a hold capacitor C1a or C1b having one terminal connected to the other terminal of the analog switch 41a or 41b and an input terminal of the voltage follower 42a or 42b at the output side, and the other terminal grounded. The voltage followers 40a and 40b and 42a and 42b function to reduce influence exerted on an input signal (i.e., the I-V-converted signal V0) and prevent discharging by load resistors. The analog switches 41a and 41b charge the hold capacitors C1a and C1b with the I-V-converted signal V0 supplied from the I-V converter 14, respectively, when turned on in response to sampling control signals SP1 and SP2, respectively, and hold voltages charged on the hold capacitors C1a and C1b, respectively, when turned off in response to the sampling control signals SP1 and SP2, respectively. That is, the first and second sample/hold circuits 15 and 16 sample/hold the I-V-converted signal V0 with timings based on the sampling control signals SP1 and SP2. The sampling control signals SP1 and SP2 have different phases, and, therefore, the first and second sample/hold circuits 15 and 16 sample/hold the I-V-converted signal V0 with different timings, which will be described later in detail. The first sample/hold circuit 15 samples/holds the I-V-converted signal V0 with the timing based on the sampling control signal SP1 and outputs the sampled/held signal as a first sampled/held signal V1. On the other hand, the second sample/hold circuit 16 samples/holds the I-V-converted signal V0 with the timing based on the sampling control signal SP2 and outputs the sampled/held signal as a second sampled/held signal V2. The first and second sampled/held signals V1 and V2 are each supplied to the subtracter 17.

The subtracter 17 includes, as shown in FIG. 5, a subtraction circuit including an operational amplification circuit 50 and resistors R1 and R2, an amplifier 51 for amplifying an output signal from the subtraction circuit, which is a result of subtraction by the subtraction circuit, and a low pass filter 52 for removing a high-frequency component from an output signal from the amplifier 51. The first sampled/held signal V1 is supplied to a non-inverting input terminal of the operational amplification circuit 50 through the resistor R1. The second sampled/held signal V2 is supplied to an inverting input terminal of the operational amplification circuit 50 through the resistor R1. The resistors R2 are connected between the non-inverting input terminal of the operational amplification circuit 50 and ground and between the inverting input terminal of the operational amplification circuit 50 and an output terminal of the operational amplification circuit 50, respectively. An output signal from the subtraction circuit with this configuration is amplified by K2 times by the amplifier 51 and a high-frequency component is removed therefrom by the low pass filter 52. As a result, the subtracter 17 performs a calculation process of (R2/R1)K2(V1−V2) with respect to the input first and second sampled/held signals V1 and V2 and outputs a result of the calculation process as a subtraction signal V3. That is, the subtracter 17 generates an output signal V3 proportional to the difference between the first sampled/held signal V1 and the second sampled/held signal V2. The subtraction signal V3 generated by the subtracter 17 is supplied to an AD converter 18.

The AD converter 18 converts the subtraction signal V3, which is an analog signal, into a digital signal in response to an AD conversion control signal ADC and outputs the converted digital signal as an AD-converted signal DT. The AD-converted signal DT generated by the AD converter 18 is supplied to an operation processing circuit 19.

The signal processing circuit 19 includes a digital signal processor (DSP) or microprocessor, etc., and performs fast Fourier transform (FFT) with respect to the supplied AD-converted signal DT to obtain a spectrum sequence of a bit signal. In this spectrum sequence, frequency corresponds to the speed of a blood cell and spectrum strength corresponds to the number of blood cells. A blood flow is a total sum of products of the speeds of respective blood cells and the number of the blood cells. Accordingly, the signal processing circuit 19 calculates the blood flow by multiplying respective spectrum sequences of bit signals by corresponding frequencies and adding up the multiplication results. The calculated blood flow is supplied to an output unit 20 through an interface circuit (not shown). The output unit 20 displays the calculated blood flow as a numeric value or graph.

A clock pulse generator 21 may include, for example, a crystal oscillator, and generates a reference clock signal CK of a stable oscillation frequency and supplies it to the timing pulse generator 22. The timing pulse generator 22 includes a frequency divider, a phase shifter, etc., and generates various control signals (SWP, SP1, SP2 and ADC) from the supplied reference clock pulse CK and supplies them to the aforementioned components, respectively. The respective components operate with timings based on the corresponding control signals supplied from the timing pulse generator 22.

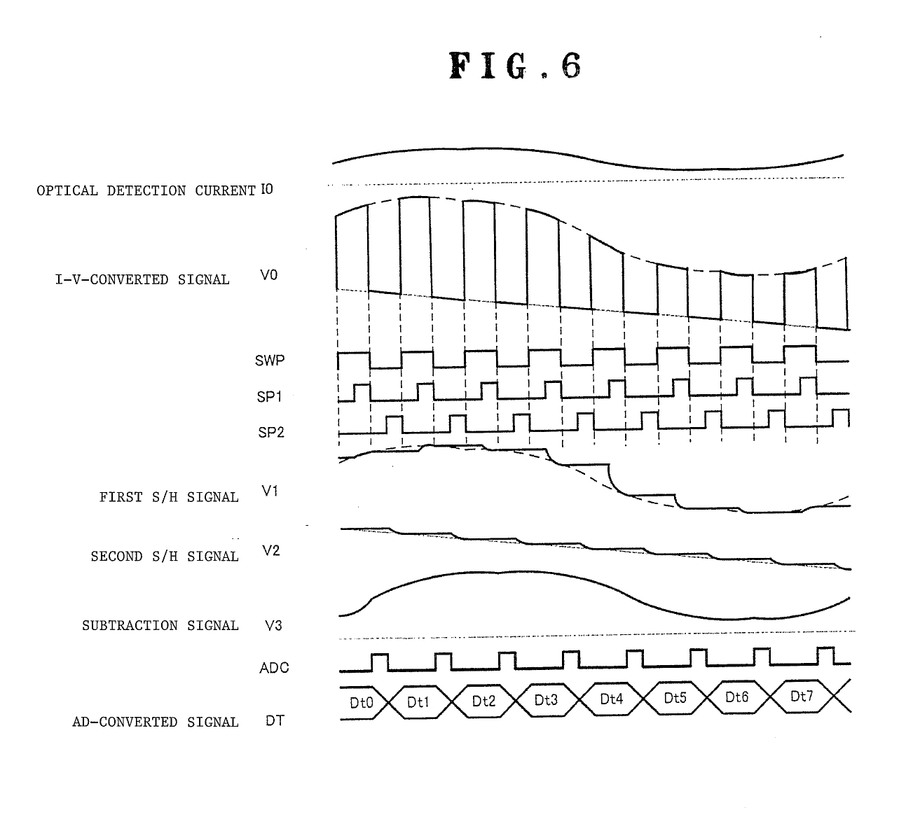

Next, the operation of the blood flow sensor with the above-stated configuration will be described with reference to a timing chart of FIG. 6. When drive current is supplied from the laser driving circuit 10, the laser light source 11 outputs laser light of power based on the drive current. The output laser light is incident onto the surface of biological tissue of a human body or the like, which is an examinee. The laser light incident on the examinee is propagated within the tissue of the examinee while being repeatedly scattered and reflected in the tissue. The scattered light reflected in the tissue is received by the photodetector 12. The photodetector 12 performs photoelectric conversion for the received scattered light to generate optical detection current I0 as a measurement signal. The optical detection current I0 is input to the switch 13.

The switch 13 is repeatedly turned on/off in response to the switch control signal SWP supplied from the timing pulse generator 22, which has a duty ratio of, for example, 50%. The optical detection current I0 is supplied to the I-V converter 14 only when the switch 13 is on. In other words, the optical detection current I0 is intermittently supplied to the I-V converter 14.

The I-V converter 14 amplifies a signal level of the optical detection current I0 by converting the optical detection current I0 into a voltage signal and amplifying the converted voltage signal. Because the optical detection current T0 is intermittently supplied by the on/off operation of the switch 13, an I-V-converted signal V0 output from the I-V converter 14 has a comb-shaped waveform as shown in FIG. 6. Since the upper envelope of the comb-shaped I-V-converted signal V0 is an amplified version of the optical detection signal I0, it conforms to the optical detection current T0, but is not completely proportional to the optical detection current T0 due to distortion. Since the lower envelope of the I-V-converted signal V0 corresponds to a period in which the optical detection signal I0 is not supplied, it conforms to a ground level, but is not completely identical to the ground level due to distortion. This is because 1/f noise generated by the operational amplification circuit 30 constituting the I-V converter 14, etc. overlap the output signal of the I-V converter 14. FIG. 6 shows an example of the case where drift-type 1/f noise falling to the right overlaps the I-V-converted signal V0. The comb-shaped I-V-converted signal V0 overlapped by this noise component is supplied to the first and second sample/hold circuits 15 and 16.

The first and second sample/hold circuits 15 and 16 sample the I-V-converted signal when the sampling control signals SP1 and SP2 are high in level, respectively, and hold the sampled signal when the sampling control signals SP1 and SP2 are low in level, respectively.

The sampling control signals SP1 and SP2 are synchronized with the switch control signal SWP. The sampling control signal SP1 assumes a high level when the switch control signal SWP is high in level, namely, when the switch 13 is conductive, and a low level when the switch control signal SWP is low in level, namely, when the switch 13 is nonconductive. Based on this sampling control signal SP1, the first sample/hold circuit 15 outputs a first sampled/held signal V1 corresponding to the upper envelope of the comb-shaped I-V-converted signal V0.

On the other hand, the sampling control signal SP2 assumes a high level when the switch control signal SWP is low in level, namely, when the switch 13 is nonconductive, and a low level when the switch control signal SWP is high in level, namely, when the switch 13 is conductive. Based on this sampling control signal SP2, the second sample/hold circuit 16 outputs a second sampled/held signal V2 corresponding to the lower envelope of the comb-shaped I-V-converted signal V0. Because the lower envelope of the I-V-converted signal V0 is generated when the switch 13 is nonconductive, namely, when the optical detection current I0 is not supplied, it does not contain a signal component and contains only a noise component. Accordingly, the second sampled/held signal V2 can be considered to be an extracted version of only the noise component overlapping the I-V-converted signal V0. The first and second sampled/held signals obtained in this manner are supplied to the subtracter 17. Preferably, as shown in FIG. 6, the sampling control signal SP1 may be adjusted for sampling in the latter half of a high-level duration of the switch control signal SWP, and the sampling control signal SP2 may be adjusted for sampling in the latter half of a low-level duration of the switch control signal SWP.

In the subtracter 17, the subtraction circuit performs a signal subtraction process to subtract the second sampled/hold voltage V2 consisting of only the noise component from the first sampled/held signal V1 corresponding to the upper envelope of the I-V-converted signal V0 containing the noise component. Then, in the subtracter 17, a result of the subtraction process is amplified by K2 times by the amplifier 51 and a high-frequency component thereof is also cut by the low pass filter 52. As a result, the subtracter 17 outputs the resulting signal as a subtraction signal V3. In other words, the subtracter 17 outputs the subtraction signal V3, which is an amplified version of the signal component alone, by removing the 1/f noise generated by the I-V converter 14 from the first sampled/held signal V1 and then amplifying and filtering the resulting signal.

The AD converter 18 AD-converts the subtraction signal V3 in response to the AD conversion control signal ADC supplied from the timing pulse generator 22 to generate an AD-converted signal DT. The AD-converted signal DT is a digital signal that is a quantized version of the signal component based on the intensity of the scattered light. The signal processing circuit 19 calculates a blood flow based on the AD-converted signal DT. The calculated blood flow is supplied to the output unit 20 through an interface circuit (not shown), and a measurement result thereof is displayed on the output unit 20 by display means of the output unit 20.

As described above, in the biological information measurement apparatus of the present invention, optical detection current I0 is intermittently supplied to the I-V converter 14, which is a 1/f noise source, by the switch 13 provided between the photodetector 12 and the I-V converter 14. As a result, the I-V converter 14 generates a comb-shaped I-V-converted signal V0 alternately having a measurement signal presence period and a measurement signal absence period. The two sample/hold circuits 15 and 16 generate a first sampled/held signal V1 obtained by intermittently sampling/holding the I-V-converted signal V0 in the measurement signal presence period, and a second sampled/held signal V2 obtained by intermittently sampling/holding the I-V-converted signal V0 in the measurement signal absence period. Because the second sampled/held signal V2 can be regarded as a noise component itself, it is possible to remove only the noise component from a measurement signal containing the noise component by subtracting the second sampled/held signal V2 from the first sampled/held signal V1. By almost completely removing the noise component from the measurement signal, it is possible to realize high precision blood flow measurement.

In the subtracter 17, the amplifier 51 performs a signal amplification process with respect to the signal from which the noise component is removed by the signal subtraction process. Therefore, it is possible to set a gain K2 to a high value without causing output saturation. Also, a detection gain before AD conversion can be set to a high value, so that a quantization error of the AD converter 18 can be reduced. In addition, the AD converter does not need to have a high resolution, thereby making it possible to reduce a bit length of the AD converter.

### Modified Embodiment 1

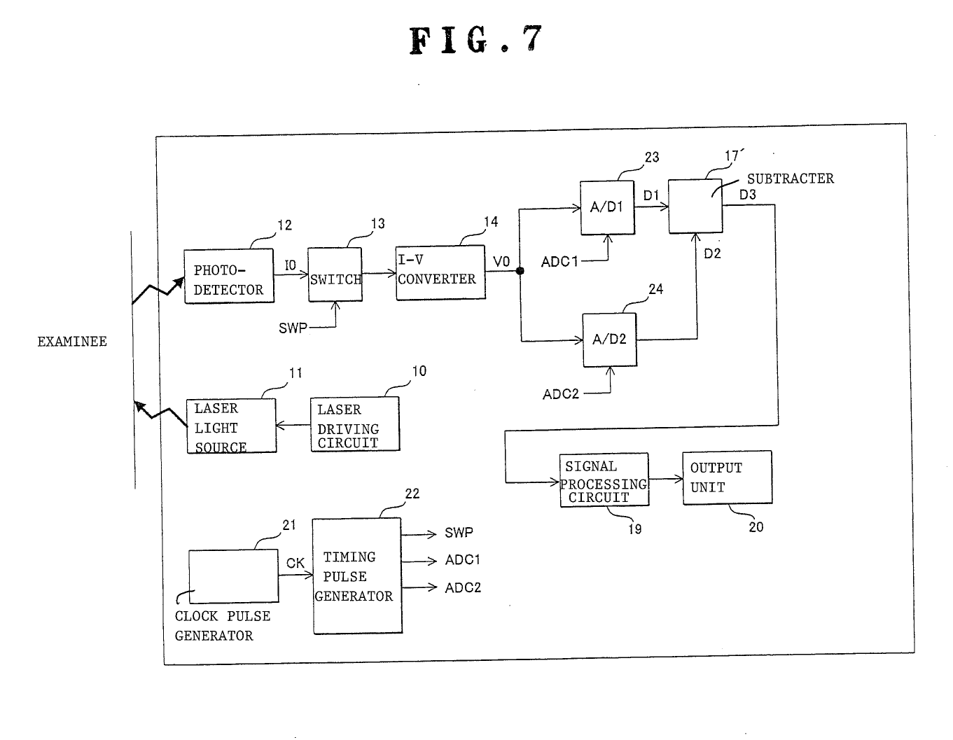

FIG. 7 is a block diagram showing the configuration of a blood flow sensor according to a modified embodiment of the present invention. The configuration of this embodiment is different from that of the above first embodiment in that the sample/hold circuits 15 and 16 according to the first embodiment are changed to AD converters 23 and 24 in the present embodiment and the AD converter 18 downstream of the subtracter 17 according to the first embodiment is deleted in the present embodiment. Also, the subtracter 17 to perform a signal calculation process for an analog signal is changed to a subtracter 17′ to perform a signal calculation process for a digital signal. Other constituent elements are the same as those of the first embodiment.

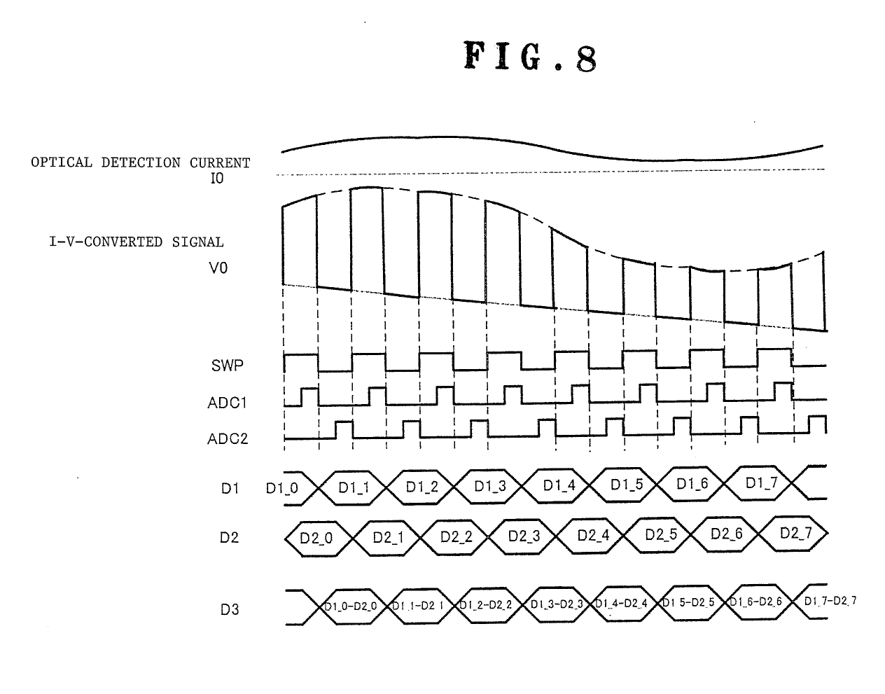

FIG. 8 is a timing chart illustrating operation timings of the respective components of the blood flow sensor according to this embodiment. The comb-shaped I-V-converted signal V0 generated by the I-V converter 14 is supplied to the first and second AD converters 23 and 24. The first and second AD converters 23 and 24 sample and quantize the I-V-converted signal V0 with timings based on AD conversion control signals ADC1 and ADC2 supplied from the timing pulse generator 22.

The AD conversion control signals ADC1 and ADC2 are synchronized with the switch control signal SWP. The AD conversion control signal ADC1 assumes a high level when the switch control signal SWP is high in level, namely, when the switch 13 is conductive, and a low level when the switch control signal SWP is low in level, namely, when the switch 13 is nonconductive. Based on this AD conversion control signal ADC1, the first AD converter 23 outputs a first AD-converted signal D1 corresponding to the upper envelope of the comb-shaped I-V-converted signal V0.

On the other hand, the AD conversion control signal ADC2 assumes a high level when the switch control signal SWP is low in level, namely, when the switch 13 is nonconductive, and a low level when the switch control signal SWP is high in level, namely, when the switch 13 is conductive. Based on this AD conversion control signal ADC2, the second AD converter 24 outputs a second AD-converted signal D2 corresponding to the lower envelope of the comb-shaped I-V-converted signal V0. Because the lower envelope of the I-V-converted signal V0 is generated when the switch 13 is nonconductive, it does not contain a signal component and contains only a noise component. Accordingly, the second AD-converted signal D2 can be considered to be an extracted version of only the noise component overlapping the I-V-converted signal V0. The first and second AD-converted signals obtained in this manner are supplied to the subtracter 17′.

The subtracter 17′ performs a signal subtraction process to subtract the second AD-converted signal D2 consisting of only the noise component from the first AD-converted signal D1 corresponding to the upper envelope of the I-V-converted signal containing the noise component, and outputs a result of the subtraction process as a subtraction signal D3. In other words, the subtracter 17′ outputs the subtraction signal D3 obtained by removing the 1/f noise generated by the I-V converter 14 from the first AD-converted signal D1. Because the subtraction signal D3 is a digital signal, it is directly supplied to the signal processing circuit 19 and then processed thereby.

As stated above, in the blood flow sensor of the configuration according to the present embodiment, it is also possible to remove only a noise component from a measurement signal overlapped by the noise component, thereby obtaining a high precision measurement result.

### Modified Embodiment 2

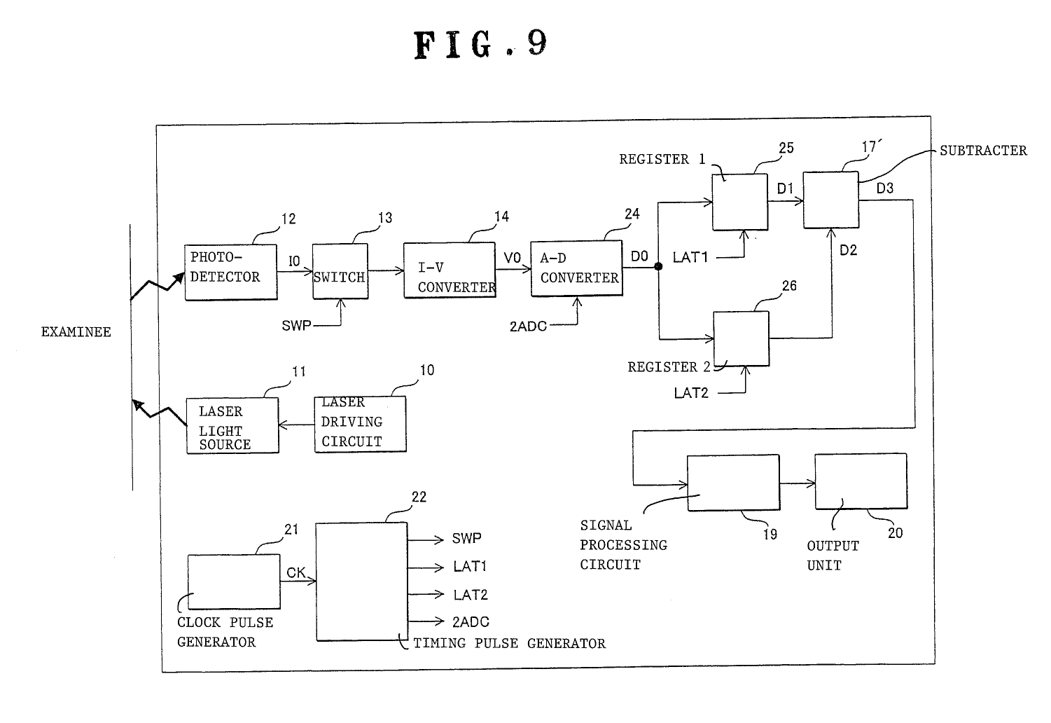

FIG. 9 is a block diagram showing the configuration of a biological information measurement apparatus according to a modified embodiment of the present invention. The configuration of this embodiment is different from that of the above first embodiment in that the sample/hold circuits 15 and 16 according to the first embodiment are changed to registers 25 and 26 in the present embodiment and the AD converter 18 downstream of the subtracter 17 according to the first embodiment is provided downstream of the I-V converter 14 in the present embodiment. Also, the subtracter 17 to perform a signal calculation process for an analog signal is changed to a subtracter 17′ to perform a signal calculation process for a digital signal. Other constituent elements are the same as those of the first embodiment.

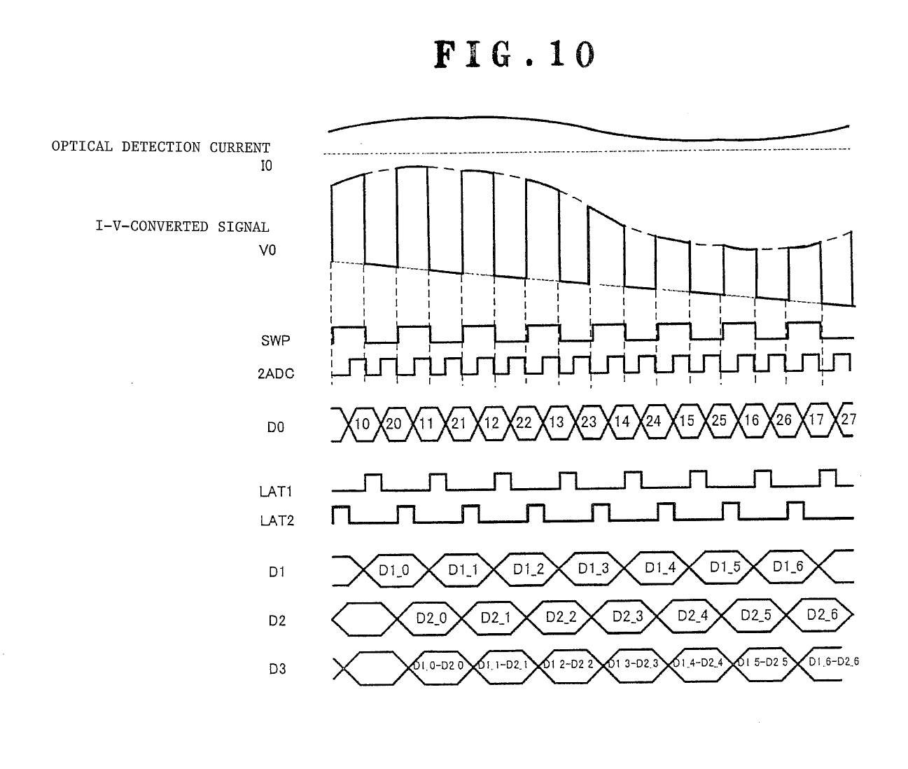

FIG. 10 is a timing chart illustrating operation timings of the respective components of the blood flow sensor according to this embodiment. The comb-shaped I-V-converted signal V0 generated by the I-V converter 14 is supplied to an AD converter 24. The converter 24 samples and quantizes the I-V-converted signal V0 with timing based on an AD conversion control signal 2ADC supplied from the timing pulse generator 22 and outputs the sampled and quantized signal as an AD-converted signal D0. The AD conversion control signal 2ADC is set to at least twice the frequency of the switch control signal SWP. By performing AD conversion based on this AD conversion control signal 2ADC, the AD converter 24 performs the AD conversion with respect to both the measurement signal presence period and measurement signal absence period of the I-V-converted signal V0. The AD-converted signal D0 is supplied to the first and second registers 25 and 26.

The first and second registers 25 and 26 hold and output the AD-converted signal D0 with timings according to which control signals LAT1 and LAT2 make low to high level transitions, respectively.

The control signal LAT1 assumes a high level with timing according to which the AD-converted output of the I-V-converted signal V0 is generated in a period in which the switch 13 is conductive, and a low level with timing according to which the AD-converted output of the I-V-converted signal V0 is generated in a period in which the switch 13 is nonconductive. Based on this control signal LAT1, the first register 25 outputs a first sampled/held signal D1 corresponding to the upper envelope of the comb-shaped I-V-converted signal V0.

On the other hand, the control signal LAT2 assumes a high level with timing according to which the AD-converted output of the I-V-converted signal V0 is generated in the period in which the switch 13 is nonconductive, and a low level with timing according to which the AD-converted output of the I-V-converted signal V0 is generated in the period in which the switch 13 is conductive. Based on this control signal LAT2, the second register 26 outputs a second sampled/held signal D2 corresponding to the lower envelope of the comb-shaped I-V-converted signal V0. Because the lower envelope of the I-V-converted signal V0 is generated When the switch 13 is nonconductive, it does not contain a signal component and contains only a noise component. Accordingly, the second sampled/held signal D2 can be considered to be an extracted version of only the noise component overlapping the I-V-converted signal V0. The first and second sampled/held signals obtained in this manner are supplied to the subtracter 17′.

The subtracter 17′ performs a signal subtraction process to subtract the second sampled/held signal D2 consisting of only the noise component from the first sampled/held signal D1 corresponding to the upper envelope of the I-V-converted signal containing the noise component, and outputs a result of the subtraction process as a subtraction signal D3. In other words, the subtracter 17′ outputs the subtraction signal D3 obtained by removing the 1/f noise generated by the I-V converter 14 from the first sampled/held signal D1. Because the subtraction signal D3 is a digital signal, it is directly supplied to the signal processing circuit 19.

As stated above, in the blood flow sensor of the configuration according to the present embodiment, it is also possible to remove only a noise component from a measurement signal overlapped by the noise component, thereby obtaining a high precision measurement result.

### Modified Embodiment 3

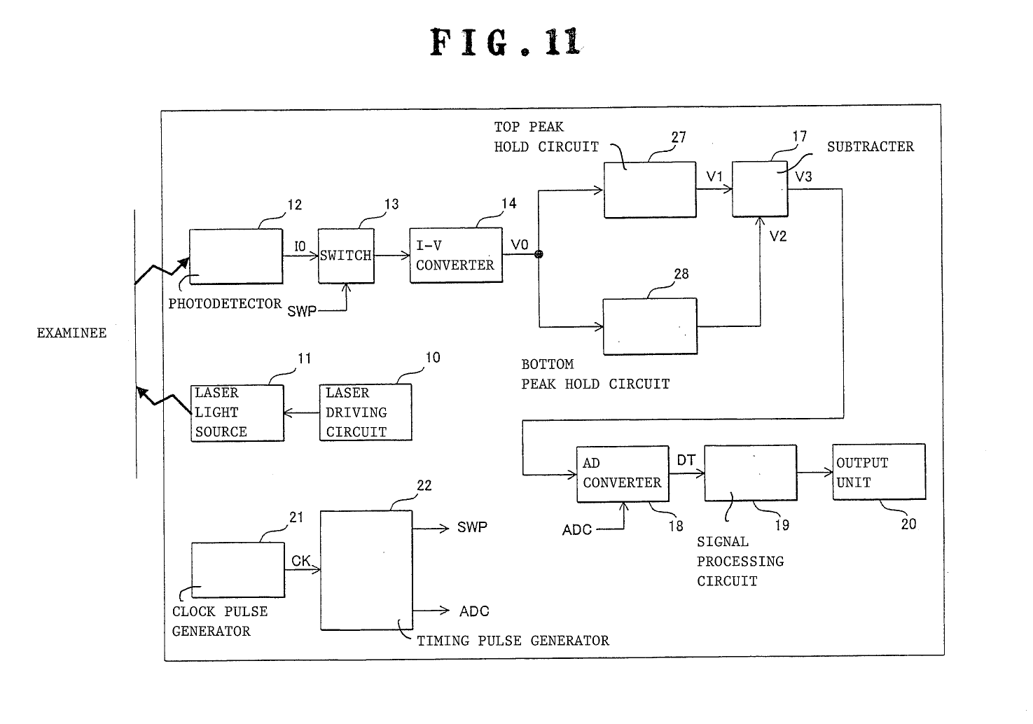

FIG. 11 is a block diagram showing the configuration of a blood flow sensor according to a modified embodiment of the present invention. The configuration of this embodiment is different from that of the above first embodiment in that the sample/hold circuits 15 and 16 according to the first embodiment are changed to a top peak hold circuit 25 and a bottom peak hold circuit 26 in the present embodiment. Other constituent elements are the same as those of the first embodiment.

The top peak hold circuit 27 detects a top peak of the input I-V-converted signal V0 within a certain time and outputs a direct current (DC) voltage identical to the detected top peak as a top peak detection signal V1. The bottom peak hold circuit 28 detects a bottom peak of the input I-V-converted signal V0 within a certain time and outputs a DC voltage identical to the detected bottom peak as a bottom peak detection signal V2. In these peak hold circuits, reset switches are provided to reset peaks held by the peak hold circuits at intervals of a predetermined period so that the peak hold circuits output a new top peak and bottom peak. These reset switches operate based on reset control signals RES1 and RES2 supplied from the timing pulse generator.

The reset control signals RES1 and RES2 are synchronized with the switch control signal SWP. The reset control signal RES1 assumes a high level when the switch control signal SWP is high in level, namely, when the switch 13 is conductive, and a low level when the switch control signal SWP is low in level, namely, when the switch 13 is nonconductive. Based on this reset control signal RES1, the top peak hold circuit 27 outputs a top peak detection signal V1 corresponding to the upper envelope of the comb-shaped I-V-converted signal V0.

On the other hand, the reset control signal RES2 assumes a high level when the switch control signal SWP is low in level, namely, when the switch 13 is nonconductive, and a low level when the switch control signal SWP is high in level, namely, when the switch 13 is conductive. Based on this reset control signal RES2, the bottom peak hold circuit 28 outputs a bottom peak detection signal V2 corresponding to the lower envelope of the comb-shaped I-V-converted signal V0. Because the lower envelope of the I-V-converted signal V0 is generated when the switch 13 is nonconductive, it does not contain a signal component and contains only a noise component. Accordingly, the bottom peak detection signal V2 can be considered to be an extracted version of only the noise component overlapping the I-V-converted signal V0. The top peak detection signal V1 and bottom peak detection signal V2 obtained in this manner are supplied to the subtracter 17.

The subtracter 17 performs a signal subtraction process to subtract the bottom peak detection signal V2 consisting of only the noise component from the top peak detection signal V1 corresponding to the upper envelope of the I-V-converted signal V0 containing the noise component. Then, in the subtracter 17, a result of the subtraction process is amplified by K2 times by the amplifier 51 and a high-frequency component thereof is also cut by the low pass filter 52. As a result, the subtracter 17 outputs the resulting signal as a subtraction signal V3. In other words, the subtracter 17 outputs the subtraction signal V3 proportional to only the signal component by removing the 1/f noise generated by the I-V converter 14 from the top peak detection signal V1 and then amplifying the resulting signal.

As stated above, in the blood flow sensor of the configuration according to the present embodiment, it is also possible to remove only a noise component from a measurement signal overlapped by the noise component, thereby obtaining a high precision measurement result.

### Modified Embodiment 5

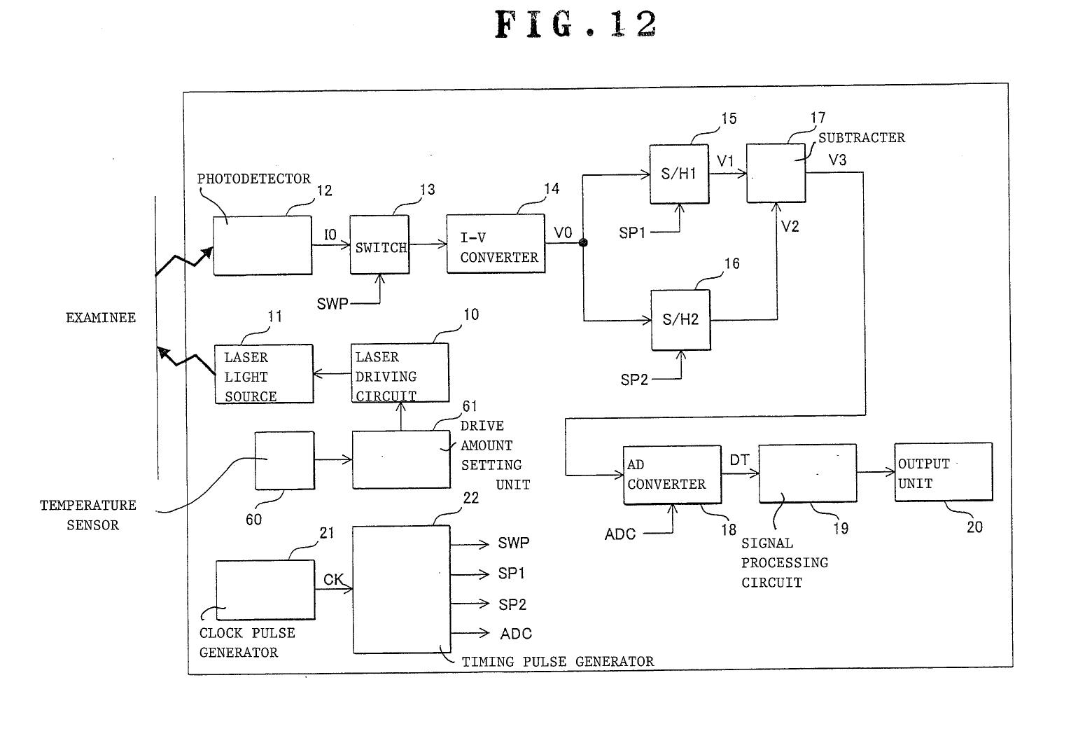

FIG. 12 is a block diagram showing the configuration of a blood flow sensor according to a modified embodiment of the present invention. The configuration of this embodiment is different from that of the above first embodiment in that a temperature sensor 60 and a drive amount setting unit 61 are further provided to adjust laser power of laser light to be emitted from the laser light source 11. Other constituent elements are the same as those of the first embodiment. The temperature sensor 60 senses an ambient temperature and supplies a temperature sense signal corresponding to the sensed temperature to the drive amount setting unit 61. The drive amount setting unit 61 includes a microcomputer, etc., and always monitors the temperature sense signal and supplies a drive command based on the temperature sense signal to the laser driving circuit 10. The drive amount setting unit 61 has a control table indicative of a corresponding relationship between the ambient temperature and the laser drive current, and generates the drive command with reference to the control table. That is, in order to correct a variation in output characteristics of the laser light source 11 with a variation in ambient temperature, the drive amount setting unit 61 sets the drive current of the laser driving circuit 10 such that laser light of constant power is output even if the ambient temperature varies. Therefore, it is possible to prevent the laser light from being projected with power of a level capable of adversely affecting the human body. Further, in a testing process before product release, a drive current-laser power characteristic of the laser light source 11 may be measured to compensate for a characteristic difference between products. For this compensation, the control table of each product may be corrected to adjust set values of the laser drive current.

### Modified Embodiment 6

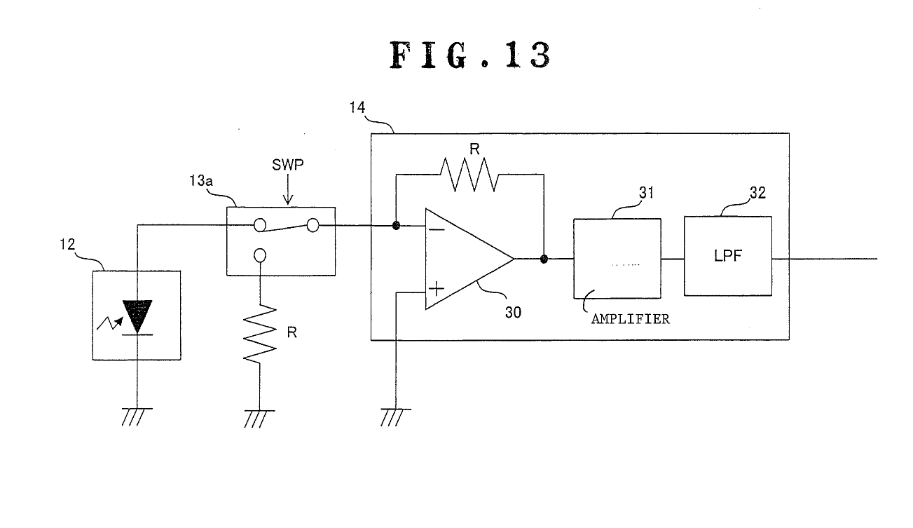

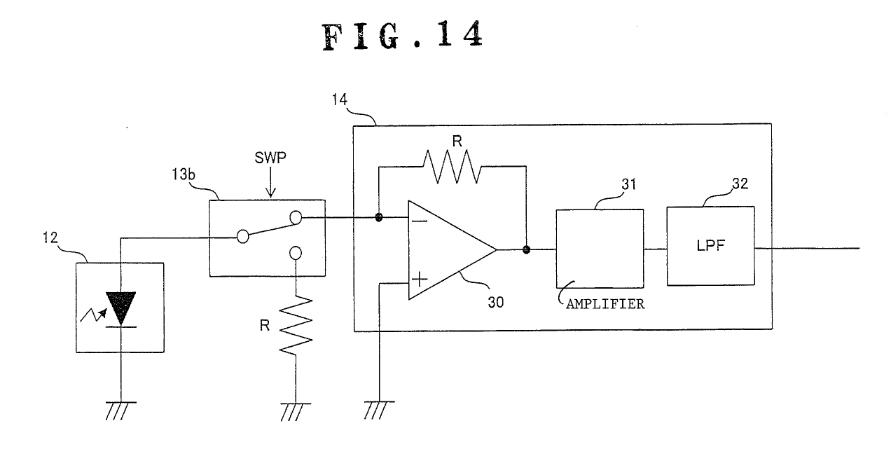

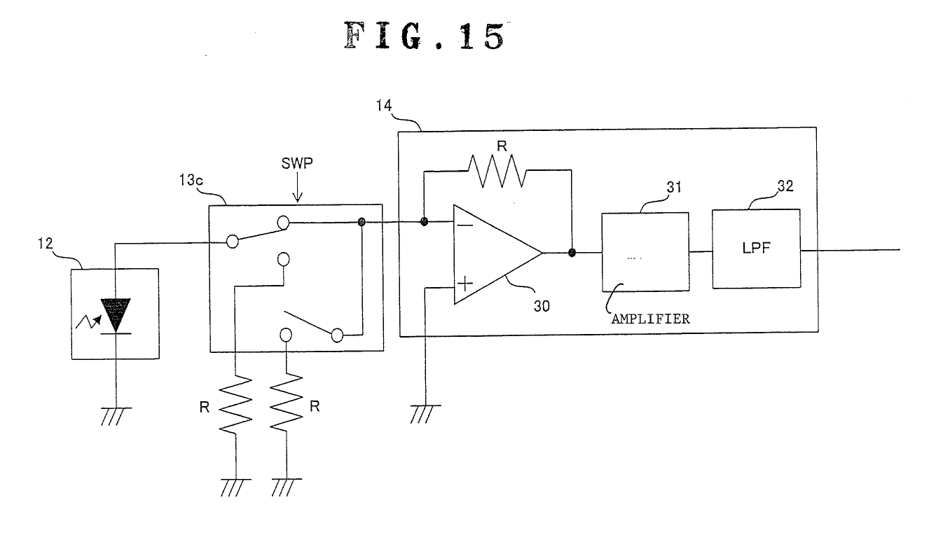

FIGS. 13 to 15 show different examples of the configuration of the switch that controls the supply/non-supply of the optical detection current I0 to the I-V converter 14. As shown in FIG. 13, a switch 13a of a 2-input 1-output selection type may be provided. In the period in which the optical detection current I0 is not supplied, the switch 13a may be switched to a resistor R to block the supply of the optical detection current I0 and ground the input terminal of the I-V converter 14 through the resistor R. Alternatively, as shown in FIG. 14, a switch 13b of a 2-input 1-output selection type may be provided. In the period in which the optical detection current I0 is not supplied, the switch 13b may be switched to a resistor R to block the supply of the optical detection current I0 and ground the output terminal of the photodetector through the resistor R. As another alternative, as shown in FIG. 15, a switch 13c may include a plurality of switch groups. In the period in which the optical detection current I0 is not supplied, the respective switches may be switched to resistors R to block the supply of the optical detection current I0 and ground both the input terminal of the I-V converter 14 and the output terminal of the photodetector through the resistors R.

### Embodiment 2

In the above first embodiment and modified embodiments thereof, the switch 13 provided between the photodetector 12 and the I-V converter 14 is turned on/off to intermittently supply the optical detection current I0, which is the measurement signal, to the I-V converter 14. In contrast, a biological information measurement apparatus according to a second embodiment of the present invention is configured to intermittently light the laser light source 11 to intermittently supply the measurement signal to the I-V converter 14. Hereinafter, the biological information measurement apparatus according to the second embodiment will be described with reference to the annexed drawings.

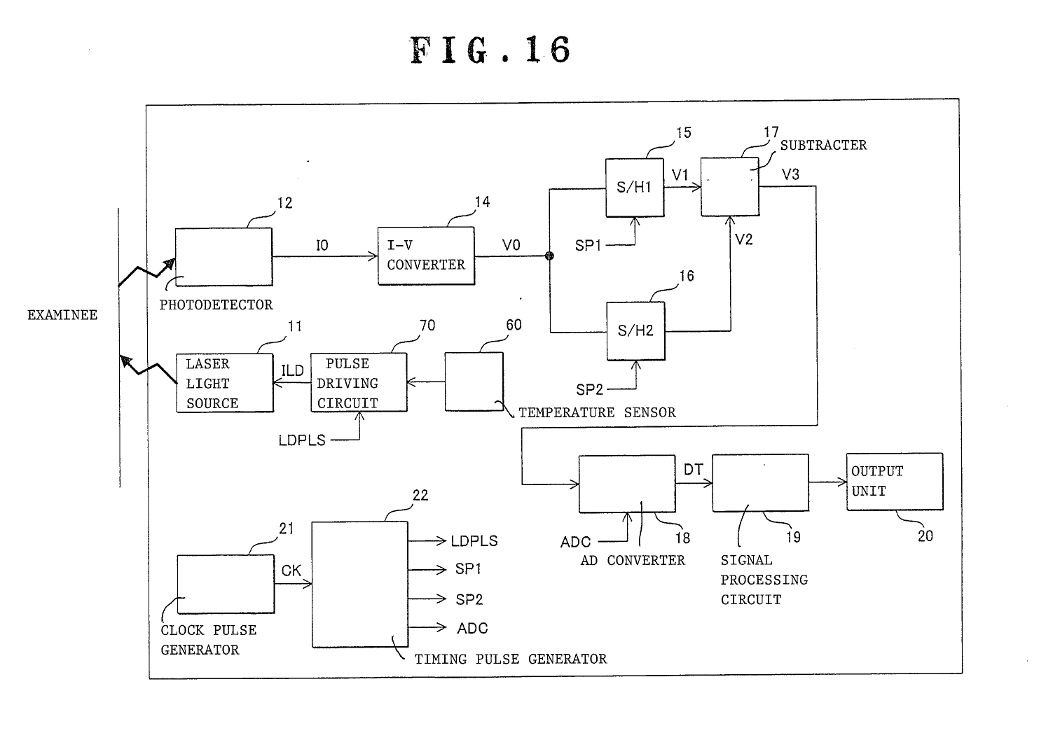

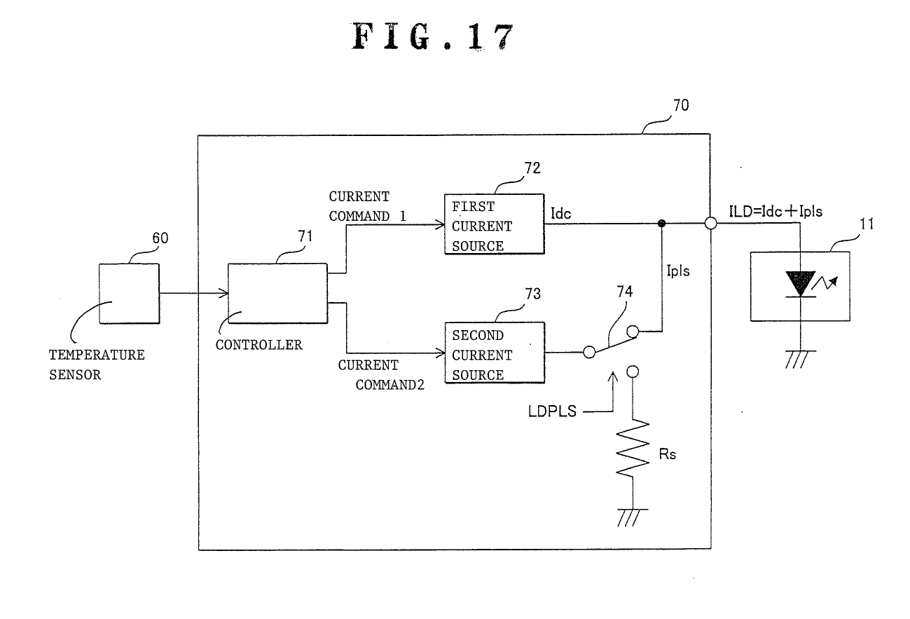

FIG. 16 is a block diagram showing the configuration of the blood flow sensor according to the second embodiment. The configuration of this embodiment is different from that of the above first embodiment in that it includes a pulse driving circuit 70 for pulse-driving the laser light source 11, and a temperature sensor 60 for sensing an ambient temperature and supplying a temperature sense signal based on the ambient temperature to the pulse driving circuit 70. Other constituent elements are the same as those of the first embodiment. FIG. 17 is a block diagram showing in detail the configuration of the pulse driving circuit 70 according to this embodiment.

A first current source 72 supplies, to the laser light source 11, reference current Idc set to a current value indicated by a current command 1 supplied from a controller 71. The reference current Idc is a DC current set to a current value in the vicinity of threshold current of the laser light source 11. A second current source 73 generates laser drive current set to a current value indicated by a current command 2 supplied from the controller 71. The laser drive current is set to a current value required for the laser light source 11 to generate desired power. A switch 74 is provided between the second current source 73 and the laser light source 11. The switch 74 is turned on/off in response to a lighting timing control signal LDPLS supplied from the timing pulse generator 22 to intermittently supply the laser drive current generated by the second current source to the laser light source 11. In other words, the pulse driving circuit 70 supplies, to the laser light source 11, laser drive current ILD obtained by adding the reference current Idc supplied from the first current source 72, which is a DC current, and pulse current Ipls of a rectangular pulse shape supplied through the switch 74 from the second current source 73.

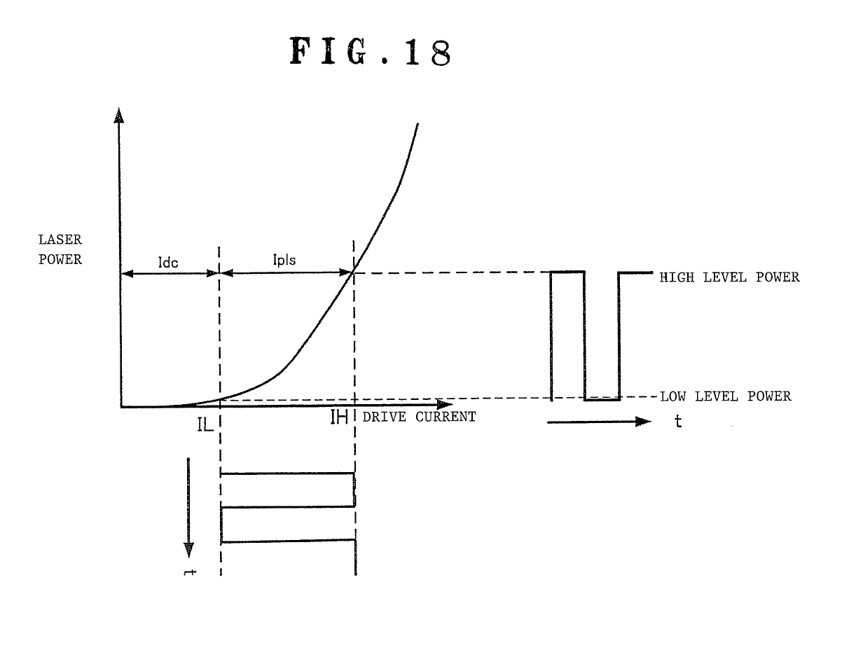

FIG. 18 illustrates a drive current to output power characteristic (I-P characteristic) of a semiconductor laser that is used for the laser light source. As shown in this drawing, in the semiconductor laser, in an area below threshold current, laser power does not rise even if drive current increases. On the other hand, in an area above threshold current, it is possible to obtain laser power which is nearly proportional to drive current. In consideration of this I-P characteristic of the semiconductor laser, the pulse driving circuit 70 according to the present embodiment has the two current sources 72 and 73, in which the first current source 11 generates the reference current Idc set to a current value in the vicinity of the threshold current and the second current source 74 generates the pulse current Ipls required to obtain a desired light emission intensity. That is, in an off period of the pulse current Ipls (namely, a period in which the switch 74 is off), only the reference current Idc is supplied to the laser light source 11. As a result, in this period, the output power of the laser light source 11 has a level close to zero (low level output), so that the laser light source 11 is extinguished. On the other hand, in an on period of the pulse current Ipls (namely, a period in which the switch 74 is on), the drive current generated by the second current source 73 is supplied to the laser light source 11 in addition to the reference current Idc. As a result, in this period, the output power of the laser light source 11 has a level required to perform measurement of a blood flow (high level output).

As stated above, the reference current Idc is always supplied to the laser light source 11 when the laser light source 11 is pulse-driven, so that the output power of the laser light source 11 can be rapidly changed from the low level power to the high level power and have an improved response characteristic with respect to the pulse input. Also, provided that on/off current increases, there is a concern that peripheral circuits could generate noise. In the present embodiment, by always supplying the reference current Idc, it is possible to make the amplitude of the pulse current Ipls in the on/off period small, thereby suppressing generation of noise.

The controller 71 includes a microcomputer, etc., and always monitors the temperature sense signal supplied from the temperature sensor 60 and supplies current commands based on the temperature sense signal to the first and second current sources 72 and 73. The controller 71 has a control table indicative of a corresponding relationship between the ambient temperature and the laser drive current, and generates the current commands with reference to the control table. By creating the control table to correct a variation in the I-P characteristic of the laser light source 11 with a variation in the ambient temperature, laser light of constant power can be output even if the ambient temperature varies. Therefore, it is possible to prevent the laser light from being projected with power of a level capable of adversely affecting the human body. Further, in a testing process before product release, a drive current-output power characteristic of the laser light source 11 may be measured to compensate for a characteristic difference between products. For this compensation, the control table of each product may be corrected to adjust set values of the laser drive current.

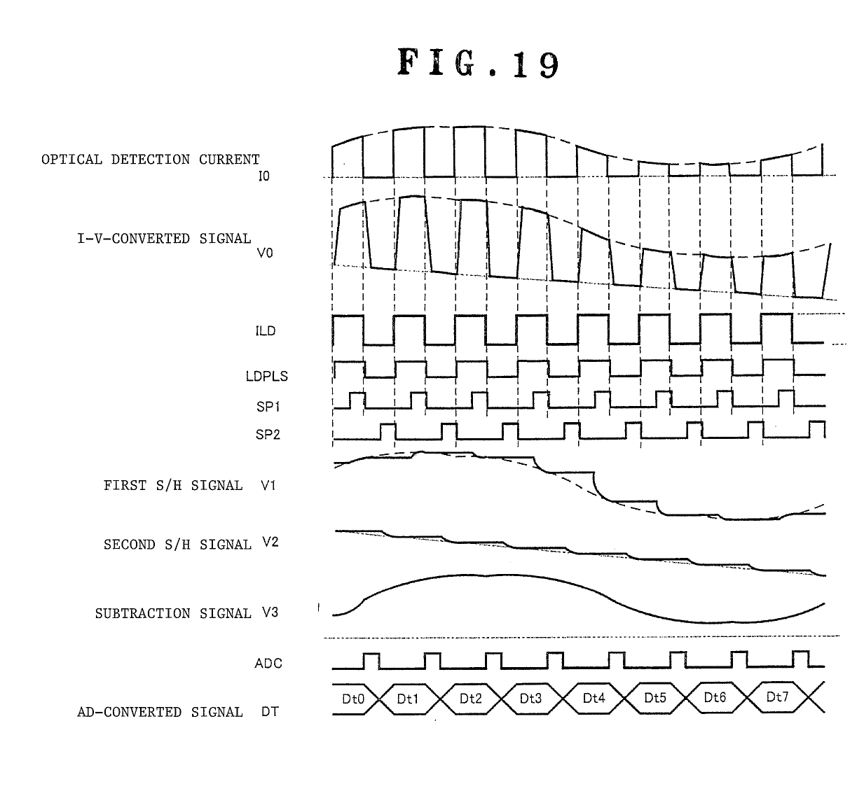

Next, the operation of the blood flow sensor according to this embodiment will be described with reference to a timing chart of FIG. 19. The switch 74 of the pulse driving circuit 70 is repeatedly turned on/off in response to the lighting timing control signal LDPLS supplied from the timing pulse generator 22, which has a duty ratio of, for example, 50%. As a result, laser drive current ILD of a rectangular pulse shape is supplied to the laser light source 11. The laser light source 11 generates laser light of high level power in a period in which laser drive current of a high level is supplied, and laser light of low level power in a period in which laser drive current of a low level is supplied. Because the laser light source 11 is almost extinguished when generating the laser light of the low level power, it is repeatedly lighted and extinguished based on the pulsed laser drive current ILD.

Scattered light generated by projecting laser light emitted from the laser light source 11 to an examinee is received by the photodetector 12. The photodetector 12 performs photoelectric conversion for the received scattered light to generate optical detection current I0. The optical detection current I0 has a comb-shaped waveform corresponding to lighting and extinction timings of the laser light source 11. That is, in a period in which the laser light source 11 is lighted, scattered light from the examinee can be received. As a result, in this period, a measurement signal can be obtained. On the other hand, in a period in which the laser light source 11 is extinguished, no scattered light from the examinee can be received. As a result, in this period, no measurement signal can be obtained. This optical detection current I0 is input to the I-V converter 14.

The I-V converter 14 amplifies a signal level of the optical detection current I0 by converting the optical detection current I0 into a voltage signal and amplifying the converted voltage signal. Because the optical detection current I0 has the comb-shaped waveform as stated above, an I-V-converted signal V0 obtained by performing current-voltage conversion with respect to the optical detection current I0 has also a waveform of the same shape. Since the upper envelope of the I-V-converted signal V0 is an amplified version of the optical detection signal I0, it conforms to the optical detection current I0, but is not completely proportional to the optical detection current I0 due to distortion. Since the lower envelope of the I-V-converted signal V0 corresponds to the extinction period of the laser light source 11, it conforms to a ground level, but is not completely identical to the ground level due to distortion. This is because 1/f noise generated by the operational amplification circuit 30 constituting the I-V converter 14, etc. overlap the output signal of the I-V converter 14. FIG. 19 shows an example of the case where drift-type noise falling to the right overlaps the I-V-converted signal V0. The comb-shaped I-V-converted signal V0 overlapped by this drift-type noise component is supplied to the first and second sample/hold circuits 15 and 16.

The first and second sample/hold circuits 15 and 16 sample the I-V-converted signal when the sampling control signals SP1 and SP2 are high in level, respectively, and hold the sampled signal when the sampling control signals SP1 and SP2 are low in level, respectively.

The sampling control signals SP1 and SP2 are synchronized with the lighting timing LDPLS. The sampling control signal SP1 assumes a high level when the lighting timing control signal LDPLS is high in level, namely, when the laser light source 11 is lighted, and a low level when the lighting timing control signal LDPLS is low in level, namely, when the laser light source 11 is extinguished. Based on this sampling control signal SP1, the first sample/hold circuit 15 outputs a first sampled/held signal V1 corresponding to the upper envelope of the comb-shaped I-V-converted signal V0.

On the other hand, the sampling control signal SP2 assumes a high level when the lighting timing control signal LDPLS is low in level, namely, when the laser light source 11 is extinguished, and a low level when the lighting timing control signal LDPLS is high in level, namely, when the laser light source 11 is lighted. Based on this sampling control signal SP2, the second sample/hold circuit 16 outputs a second sampled/held signal V2 corresponding to the lower envelope of the comb-shaped I-V-converted signal V0. Because the lower envelope of the I-V-converted signal V0 is generated when the laser light source 11 is extinguished, it does not contain a signal component and contains only a noise component. Accordingly, the second sampled/held signal V2 can be considered to be an extracted version of only the noise component overlapping the I-V-converted signal V0.

The first and second sampled/held signals obtained in this manner are supplied to the subtracter 17. Preferably, as shown in FIG. 19, the sampling control signal SP1 may be adjusted for sampling in the latter half of a high-level duration of the lighting timing control signal LDPLS, and the sampling control signal SP2 may be adjusted for sampling in the latter half of a low-level duration of the lighting timing control signal LDPLS.

In the subtracter 17, the subtraction circuit performs a signal subtraction process to subtract the sampled/hold voltage V2 consisting of only the noise component from the sampled/held signal V1 corresponding to the upper envelope of the I-V-converted signal V0 containing the noise component. Then, in the subtracter 17, a result of the subtraction process is amplified by K2 times by the amplifier 51 and a high-frequency component thereof is also cut by the low pass filter 52. As a result, the subtracter 17 outputs the resulting signal as a subtraction signal V3. In other words, the subtracter 17 outputs the subtraction signal V3 proportional to only the signal component by removing the 1/f noise generated by the I-V converter 14 from the sampled/held signal V1 and then amplifying the resulting signal.

The AD converter 18 AD-converts the subtraction signal V3 in response to the AD conversion control signal ADC supplied from the timing pulse generator 22 to generate an AD-converted signal DT, which is discrete data that is a quantized version of the signal component based on the intensity of the scattered light. The signal processing circuit 19 calculates a blood flow based on the AD-converted signal DT. The calculated blood flow is supplied to the output unit 20 through an interface circuit (not shown), and a measurement result thereof is displayed on the output unit 20 by display means of the output unit 20.

As described above, in the biological information measurement apparatus of the second embodiment, the laser light source 11 is pulse-driven, thereby generating a comb-shaped I-V-converted signal V0 alternately having a measurement signal presence period and a measurement signal absence period. The two sample/hold circuits 15 and 16 generate a first sampled/held signal V1 obtained by intermittently sampling/holding the I-V-converted signal V0 in the measurement signal presence period, and a second sampled/held signal V2 obtained by intermittently sampling/holding the I-V-converted signal V0 in the measurement signal absence period. Because the second sampled/held signal V2 can be regarded as a noise component itself, it is possible to remove only the noise component from a detection signal overlapped by the noise component by subtracting the second sampled/held signal V2 from the first sampled/held signal V1. Therefore, similarly to the first embodiment, it is possible to obtain a high precision measurement result.

Also, in the present embodiment, because the laser light source 11 is pulse-driven, it is possible to reduce power consumption as compared with the case where the laser irradiation is performed with only power of a high level. Also, because the apparatus can operate with low power consumption, it may be driven by a battery, thereby making it possible to implement a compact apparatus with excellent portability. Also, although the above embodiment has been configured to always supply reference current Idc, drive current may be set to zero when the laser light source 11 is extinguished, in order to reduce power consumption still further. In addition, power consumption may be reduced still further by making duty ratios in the lighting period and extinction period small.

### Modified Embodiment

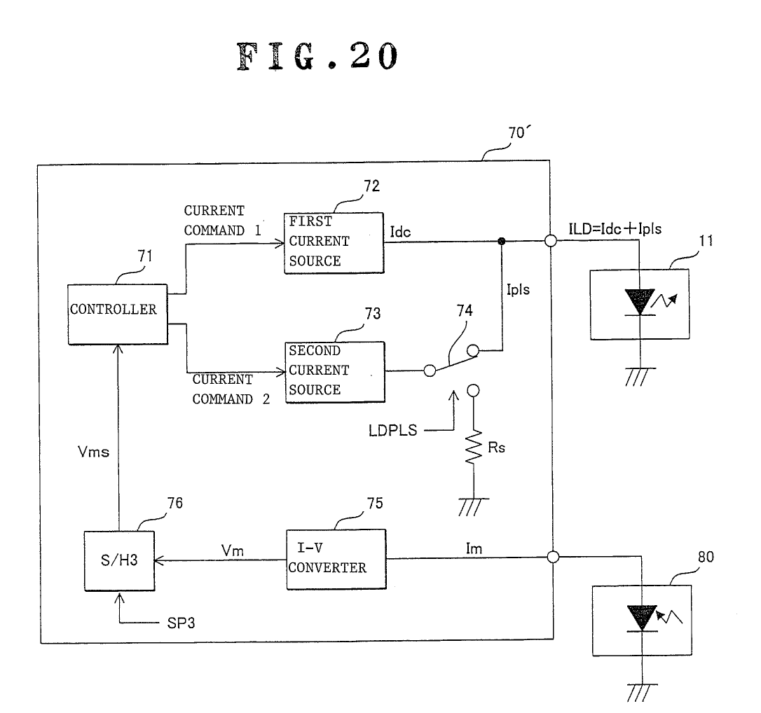

FIG. 20 is a block diagram showing the configuration of a pulse driving circuit 70′ according to a modified embodiment of the present invention, which is a modification of the pulse driving circuit 70. The control of the output power of the laser light by the pulse driving circuit 70 according to the second embodiment is performed in a feedforward manner. In contrast, in the present embodiment, the pulse driving circuit 70′ performs a negative feedback control to prevent a variation in the output power of the laser light resulting from a temperature, etc.

A photodetector 80 for output monitor is disposed to directly receive a part of the laser light emitted from the laser light source 11. The output monitor photodetector 80 performs photoelectric conversion for the received light to generate monitor current Im based on the amount of the received light. An I-V converter 75 converts the monitor current Im into a voltage signal, amplifies the voltage signal and outputs the amplified signal as an I-V-converted signal Vm. A sample/hold circuit 76 samples/holds the I-V-converted signal Vm with timing based on a sampling control signal SP3 supplied from the timing pulse generator 22 and outputs the sampled/held signal as a sampled/held signal Vms. The sampling control signal SP3 is adjusted in timing to sample/hold the I-V-converted signal Vms when the laser light source 11 is lighted. Based on this sampling control signal SP3, the sample/hold circuit 76 outputs the sampled/held signal Vms proportional to the output power of the laser light source 11.

The controller 71 integrates an error between the present output power of the laser light source 11 indicated by the sampled/held signal Vms and target output power prestored in an internal memory and generates a current command to make the error zero. Then, each of the first and second current sources 72 and 73 generates drive current based on the current command generated by the controller 71 and supplies it to the laser light source 11. Alternatively, the drive current control may be applied to only the second current source 73 that determines the output power of the laser light source 10.

As stated above, by forming a closed loop by the monitor photodetector 80, I-V converter 75, sample/hold circuit 76, controller 71, first and second current sources 72 and 73 and laser light source 11 and executing the negative feedback control, it is possible to maintain the output power of the laser light source 11 constant irrespective of variations in a temperature, etc.

As is apparent from the above description, in a biological information measurement apparatus of the present invention, a measurement signal based on scattered light is intermittently supplied to an I-V converter, which is a noise source, thereby generating an I-V-converted signal having a portion corresponding to a measurement signal supply period and a portion corresponding to a measurement signal non-supply period. The upper envelope of the I-V-converted signal corresponding to the measurement signal supply period and the lower envelope of the I-V-converted signal corresponding to the measurement signal non-supply period are individually extracted and then subtracted from each other, so that a noise component is removed from the I-V-converted signal and only a signal component is thus extracted from the I-V-converted signal. Therefore, it is possible to improve measurement precision and solve the problem of output saturation in processing the measurement signal by an internal circuit. Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A biological information measurement apparatus for projecting laser light on an examinee and measuring a state of internal tissue of the examinee based on light scattered within the examinee, the apparatus comprising:
a laser light source for emitting the laser light;
photoelectric conversion means for receiving the scattered light and generating a measurement signal based on the scattered light;
signal amplification means for generating an amplified signal by amplifying a signal level of the measurement signal;
signal supply means for intermittently supplying the measurement signal to the signal amplification means;
first output means for sampling the amplified signal corresponding to a period in which the measurement signal is supplied to the signal amplification means and outputting the sampled signal as a first signal;
second output means for sampling the amplified signal corresponding to a period in which the measurement signal is not supplied to the signal amplification means and outputting the sampled signal as a second signal;
signal subtraction means for generating a subtraction signal based on a difference between the first signal and the second signal; and
arithmetic output means for arithmetically outputting information about the internal tissue of the examinee based on the subtraction signal,
wherein the signal supply means comprises a switch provided between the photoelectric conversion means and the signal amplification means, the switch being turned on/off corresponding to the period in which the measurement signal is supplied to the signal amplification means and the period in which the measurement signal is not supplied to the signal amplification means.

2. (canceled)

3. (canceled)

4. The biological information measurement apparatus according to claim 1, wherein the first and second output means comprise sample/hold circuits for holding and outputting the amplified signal synchronously with the period in which the measurement signal is supplied to the signal amplification means and the period in which the measurement signal is not supplied to the signal amplification means.

5. The biological information measurement apparatus according to claim 1, wherein the first and second output means comprise analog/digital (AD) converters for AD-converting and outputting the amplified signal synchronously with the period in which the measurement signal is supplied to the signal amplification means and the period in which the measurement signal is not supplied to the signal amplification means.

6. The biological information measurement apparatus according to claim 1, wherein:
the first output means comprises a top peak hold circuit for detecting and outputting a top peak of the amplified signal within a certain period; and
the second output means comprises a bottom peak hold circuit for detecting and outputting a bottom peak of the amplified signal within a certain period.

7. The biological information measurement apparatus according to claim 1, further comprising an amplification circuit for amplifying the subtraction signal.

8. The biological information measurement apparatus according to claim 1, further comprising an AD converter for AD-converting any one of the amplified signal or the subtraction signal.

9. (canceled)

10. (canceled)

11. (canceled)

12. (canceled)

13. A biological information measurement apparatus for projecting laser light on an examinee and measuring a state of internal tissue of the examinee based on light scattered within the examinee, the apparatus comprising:
a laser light source for emitting the laser light;
photoelectric conversion means for receiving the scattered light and generating a measurement signal based on the scattered light;
signal amplification means for generating an amplified signal by amplifying a signal level of the measurement signal;
signal supply means for intermittently supplying the measurement signal to the signal amplification means;
first output means for sampling the amplified signal corresponding to a period in which the measurement signal is supplied to the signal amplification means and outputting the sampled signal as a first signal;
second output means for sampling the amplified signal corresponding to a period in which the measurement signal is not supplied to the signal amplification means and outputting the sampled signal as a second signal;
signal subtraction means for generating a subtraction signal based on a difference between the first signal and the second signal; and
arithmetic output means for arithmetically outputting information about the internal tissue of the examinee based on the subtraction signal,
wherein the signal supply means comprises a laser driving circuit for intermittently lighting the laser light source corresponding to the period in which the measurement signal is supplied to the signal amplification means and the period in which the measurement signal is not supplied to the signal amplification means,
wherein the laser driving circuit comprises:
first drive current supply means for supplying direct current (DC) drive current to the laser light source; and
second drive current supply means for supplying pulsed drive current to the laser light source.

14. The biological information measurement apparatus according to claim 13, further comprising a temperature sensor for generating a temperature sense signal based on an ambient temperature,
wherein the laser driving circuit supplies drive current of a current value based on the temperature sense signal to the laser light source.

15. The biological information measurement apparatus according to claim 13, further comprising light receiving means for receiving a part of the laser light and generating an optical detection signal based on an emission intensity of the laser light,
wherein the laser driving circuit supplies drive current to the laser light source such that a signal level of the optical detection signal becomes a desired value.

16. A biological information measurement apparatus for projecting laser light on an examinee and measuring a state of internal tissue of the examinee based on light scattered within the examinee, the apparatus comprising:
a laser light source which emits the laser light;
a photoelectric conversion part which receives the scattered light and generating a measurement signal based on the scattered light;
a signal amplification part which generates an amplified signal by amplifying a signal level of the measurement signal;
a signal supply part which intermittently supplies the measurement signal to the signal amplification part;
a first output part which samples the amplified signal corresponding to a period in which the measurement signal is supplied to the signal amplification part and outputs the sampled signal as a first signal;
a second output part which samples the amplified signal corresponding to a period in which the measurement signal is not supplied to the signal amplification part and outputs the sampled signal as a second signal;
a signal subtraction part which generates a subtraction signal based on a difference between the first signal and the second signal; and
an arithmetic output part which arithmetically outputs information about the internal tissue of the examinee based on the subtraction signal,
wherein the signal supply part comprises a switch provided between the photo electric conversion part and the signal amplification part, the switch being turned on/off corresponding to the period in which the measurement signal is supplied to the signal amplification part and the period in which the measurement signal is not supplied to the signal amplification part.

17. The biological information measurement apparatus according to claim 16, wherein the first and second output part comprise sample/hold circuits which hold and output the amplified signal synchronously with the period in which the measurement signal is supplied to the signal amplification part and the period in which the measurement signal is not supplied to the signal amplification part.

18. The biological information measurement apparatus according to claim 16, wherein the first and second output parts comprise analog/digital (AD) converters which AD-convert and output the amplified signal synchronously with the period in which the measurement signal is supplied to the signal amplification part and the period in which the measurement signal is not supplied to the signal amplification part.

19. The biological information measurement apparatus according to claim 16, wherein:
the first output part comprises a top peak hold circuit which detects and outputs a top peak of the amplified signal within a certain period; and
the second output part comprises a bottom peak hold circuit which detects and outputs a bottom peak of the amplified signal within a certain period.

20. The biological information measurement apparatus according to claim 16, further comprising an amplification circuit which amplifies the subtraction signal.

21. The biological information measurement apparatus according to claim 16, further comprising an AD converter which AD-converts any one of the amplified signal or the subtraction signal.

22. A biological information measurement apparatus for projecting laser light on an examinee and measuring a state of internal tissue of the examinee based on light scattered within the examinee, the apparatus comprising:
a laser light source which emits the laser light;
a photoelectric conversion part which receives the scattered light and generating a measurement signal based on the scattered light;
a signal amplification part which generates an amplified signal by amplifying a signal level of the measurement signal;
a signal supply part which intermittently supplies the measurement signal to the signal amplification part;
a first output part which samples the amplified signal corresponding to a period in which the measurement signal is supplied to the signal amplification part and outputs the sampled signal as a first signal;
a second output part which samples the amplified signal corresponding to a period in which the measurement signal is not supplied to the signal amplification part and outputting the sampled signal as a second signal;
a signal subtraction part which generates a subtraction signal based on a difference between the first signal and the second signal; and
an arithmetic output part which arithmetically outputs information about the internal tissue of the examinee based on the subtraction signal,
wherein the signal supply part comprises a laser driving circuit which intermittently lights the laser light source corresponding to the period in which the measurement signal is supplied to the signal amplification part and the period in which the measurement signal is not supplied to the signal amplification part,
wherein the laser driving circuit comprises:
a first drive current supply part which supplies a direct current (DC) drive current to the laser light source; and
a second drive current supply part which supplies a pulsed drive current to the laser light source.

23. The biological information measurement apparatus according to claim 22, further comprising a temperature sensor which generates a temperature sense signal based on an ambient temperature,
wherein the laser driving circuit supplies drive current of a current value based on the temperature sense signal to the laser light source.

24. The biological information measurement apparatus according to claim 22, further comprising a light receiving part which receives a part of the laser light and generating an optical detection signal based on an emission intensity of the laser light,
wherein the laser driving circuit supplies drive current to the laser light source such that a signal level of the optical detection signal becomes a desired value.
